(19) United States
(12) Patent Application Publication
(10) Pub. No.: US 2005/0054084 A1
(43) Pub. Date: Mar. 10, 2005

(54) Substrate with at least one pore

(75) Inventor: Volker Lehmann, Munchen (DE)

Correspondence Address: Jeffrey R. Stone;Altera Law Group, Suite 100, 6500 City West Parkway, Minneapolis, MN, 55344-7704, US

(73) Assignee: Infineon Technologies AG

(21) Appl. No.: 10/942,483

(22) Filed: Sep. 16, 2004

Related U.S. Application Data
(62) Division of application No. 10/110,016, filed on Jun. 19, 2002.

(30) Foreign Application Priority Data
Oct. 6, 1999 (DE) 199 48 089.3

(51) Int. Cl. C12Q 1/68; C12M 1/34

(52) U.S. Cl. 435/287.2; 435/6

(57) ABSTRACT

A surface of a pore (P) is provided with a coating for reflecting electromagnetic radiation. The coating comprises one or more overlapping layers (A1, B1). If the surface (1) consists of silicon, the layers (A1, B1) are preferably made of materials that can be deposited in the course of CVD-processes. The layers (A1, B1) are, for example, comprised of SiO₂, polysilicon, silicon nitride or tungsten. The substrate (1) is, for example, provided as a component of a biochip used to detect fluorescing molecules (M) applied to the surface of pores (P) provided with said coating. The substrate (1) can be part of a fibre-optic light guide. The coating is preferably constructed in such a way that electromagnetic radiation having a wavelength between 400 nm and 700 nm is optimally reflected.

A biochip can be used to examine a solution of DNA sequences with regard to the presence of particular DNA sequences. To that end, for each DNA sequence to be detected, DNA sequences complementary to it are produced, applied to a region of a substrate of the biochip and immobilized by means of an adhesion layer. Each DNA sequence to be detected has a different region of the substrate allocated to it. Each DNA sequence in the solution is bonded to a fluorescent molecule by a chemical process. The solution is then applied to the substrate of the biochip. Out of the solution of DNA sequences, only the DNA sequences to be detected bind to the respectively complementary DNA sequences. After the rest of the solution has been removed, the substrate is exposed to light and a measurement is taken of whether, and from which regions of the substrate, light is emitted by the fluorescent molecules. Since a particular DNA sequence to be detected is allocated to each region, it is possible to determine not only whether DNA sequences to be detected are present in the solution, but also which of the DNA sequences to be detected are present in the solution.

The company Affimetrix markets biochips which have substrates with planar surfaces.

In order to increase the detection sensitivity for the DNA sequences to be detected, and therefore the fluorescent molecules, U.S. Pat. No. 5,843,767 proposes to use a porous glass or silicon substrate as the substrate of a biochip. The pores increase the effective surface area of the substrate, so that larger regions of the substrate can be provided with the complementary DNA sequences, which can consequently bind more DNA sequences to be detected, and this increases the amount of light emitted by fluorescence per region, i.e. per DNA sequence to be detected. So that it is particularly easy for the solution to come into contact with all of the effective surface area of the substrate, the pores extend from one surface of the substrate as far as a surface of the substrate lying opposite the surface. The solution can flow through the pores, and can hence come into contact with surfaces of the pores.

It is an object of the invention to provide a substrate with at least one pore, which is suitable as part of a biochip with a detection sensitivity for fluorescent molecules that is increased compared with the prior art.

The object is achieved by a substrate with at least one pore, in which at least one surface of the pore is provided with a coating for reflecting electromagnetic radiation.

The substrate may have further pores, which are configured like the pore. The substrate may be part of a biochip for detecting fluorescent molecules applied to the surfaces of the pores provided with the coating.

By virtue of the coating, less of the electromagnetic radiation which is emitted through fluorescence by the fluorescent molecules, and which is measured during the detection of the fluorescent molecules, is lost on the way to the exit from the pores. The emitted radiation is reflected more strongly and absorbed less inside the pores owing to the coating, so that the amount of measured electromagnetic radiation is increased, and the detection sensitivity is consequently increased.

To make it particularly easy for a solution of fluorescent molecules to reach all of the surface of the pore, the pore preferably extends from one surface of the substrate as far as a surface of the substrate lying opposite the surface. The solution may be pumped through the pores.

The substrate may also be used in a way other than for detecting fluorescent molecules. For example, the substrate is an optical waveguide, or part of an optical waveguide, which conducts light from one end of the pore to the other end of the pore by reflection. Compared with a glass fiber, such an optical waveguide has the advantage that, when entering and when exiting, light does not have to pass through any interface between a material of the optical waveguide and air, such an interface generally being reflective and consequently reducing the light intensity.

If the substrate is part of a biochip, in which fluorescent molecules that emit electromagnetic radiation with wavelengths between 700 nm and 400 mm are to be detected, then the coating is preferably configured in such a way that it optimally reflects electromagnetic radiation with precisely these wavelengths.

If the electromagnetic radiation is reflected many times inside the pore before leaving the pore, then its intensity will be commensurately smaller. Since the reflectivity of the coating also depends on the angle of incidence of the electromagnetic radiation, the coating can be optimized for particular angles of incidence. The coating is preferably optimized for angles of incidence at which the electromagnetic radiation is reflected only about three or four times on average. In this case, the optimization also depends on the dimensions of the pore, such as its depth and its diameter.

The aperture of the converging optics for detecting the emitted radiation, as well as the distance thereof from the substrate, may also be taken into account during the optimization. The converging optics, which comprise optical lenses, can only receive radiation that has been emitted at an angle which is greater than a minimum angle dependent on the distance. The optimization is preferably carried out for such angles.

Further, the refractive index of the medium located in the pore, and interlayers possibly present between the substrate and the converging optics, may also be taken into account during the optimization. The wavelength of the stimulating electromagnetic radiation may also be taken into account.

The coating is preferably configured in such a way that it optimally reflects electromagnetic radiation that strikes the coating at an angle of incidence of between 50° and 90°. In particular, this angle of incidence may be about 70°.

The substrate consists, for example, of glass.

The substrate preferably consists of silicon. Silicon is easier to structure than glass. Further, electromagnetic radiation from different pores, which belong to regions of the substrate that are provided with different complementary DNA sequences, is mixed together negligibly in comparison with glass, since silicon is opaque to electromagnetic radiation over a large frequency range.

If the substrate consists of silicon, then the coating is preferably configured in such a way that it can be fabricated by using standard processes from microelectronics.

The coating may, for example, consist of a single layer.

The coating has a particularly good reflectivity when it contains metal. The coating preferably contains tungsten, since a CVD process can be used to apply tungsten even to surfaces of pores that are deeper than 10 μm and also have a diameter less than approximately 1000 nm in size.

A dielectric layer whose dielectric constant is greater than that of the substrate is also suitable as the coating.

To increase the reflectivity, the coating may also consist of at least two layers arranged above one another. Preferably, the layers alternately have a high dielectric constant and a low dielectric constant.

Preferably, at least one of the layers consists of SiO₂, polysilicon or silicon nitride, since these materials can be produced by using standard processes from microelectronics.

Preferably, the outermost layer of the coating, i.e. the layer which the electromagnetic radiation encounters first, consists of SiO₂ or polysilicon, since methods with which the complementary DNA sequences can be fixed to these materials are known.

Preferably, all the layers of the coating consist of SiO₂, polysilicon or silicon nitride.

A high reflectivity, together with low process outlay, can be achieved if the coating consists of a first layer, which is arranged on the substrate, and a second layer arranged on the first layer.

Preferably, the first layer consists of SiO₂ or silicon nitride. The second layer preferably consists of polysilicon.

A better reflectivity is achieved if the coating consists of a first layer, which is arranged on the substrate, a second layer arranged on the first layer, and a third layer arranged on the second layer.

For example, the first layer and the third layer consist of silicon nitride or SiO₂. The second layer consists, for example, of polysilicon.

An even higher reflectivity is achieved if the coating consists of a first layer, which is arranged on the substrate, a second layer arranged on the first layer, a third layer arranged on the second layer, and a fourth layer arranged on the third layer.

The first layer and the third layer consist, for example, of silicon nitride or SiO₂. The second layer and the fourth layer consist, for example, of polysilicon.

During the optimization of the coating, the thicknesses of its layers are matched to the respective requirements.

Exemplary embodiments of the invention will be explained in more detail below with reference to the figures.

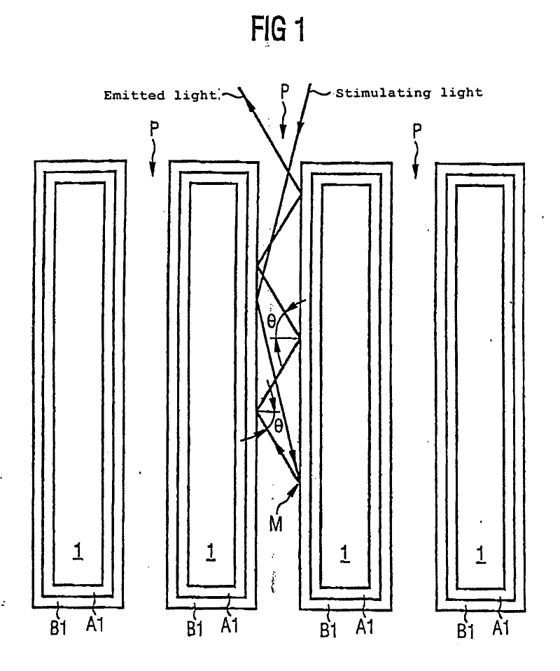

FIG. 1 shows a cross section through a first substrate with pores, a first layer and a second layer. The path of light that stimulates a fluorescent molecule, and the path of light emitted by the fluorescent molecule, are further represented.

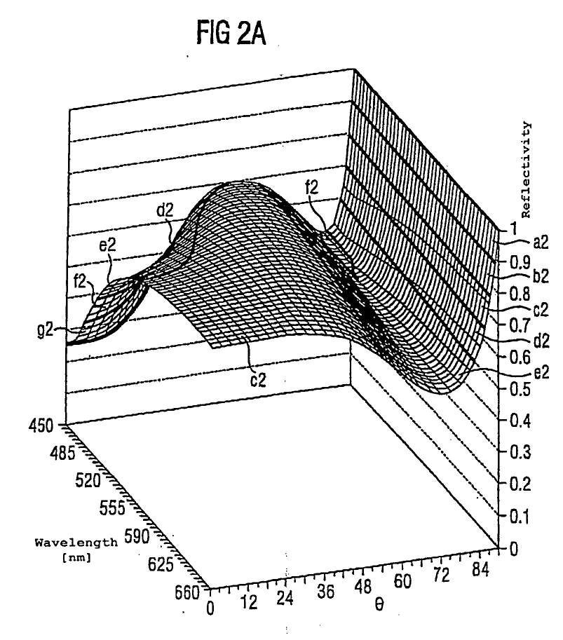

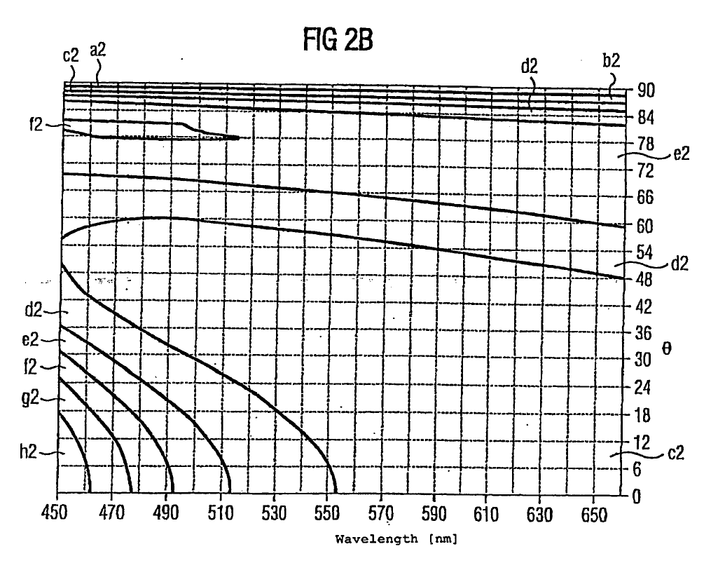

FIG. 2a shows the dependency of the reflectivity of the coated first substrate as a function of the angle of incidence and the wavelength of the electromagnetic radiation to be reflected, in a three-dimensional representation.

FIG. 2b shows the dependency from FIG. 2a in a two-dimensional representation.

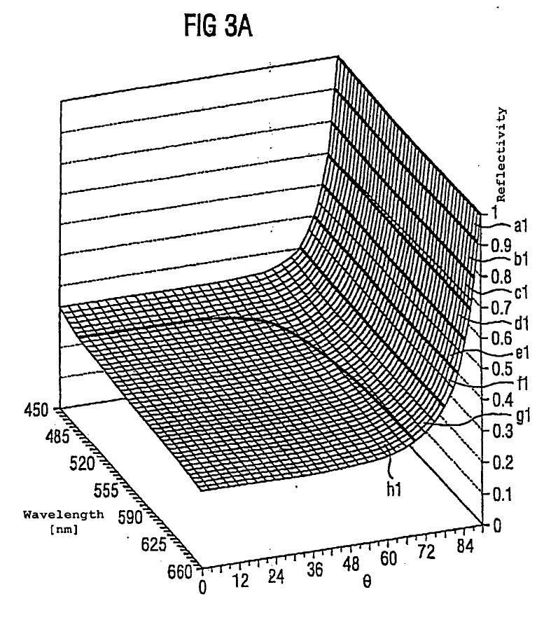

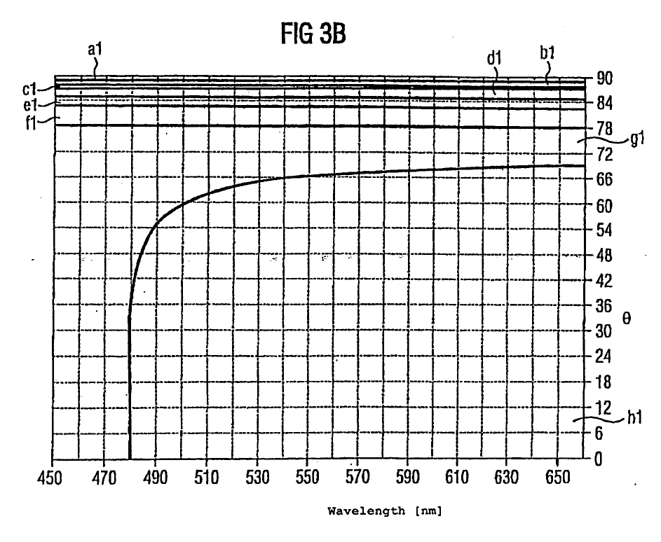

FIG. 3a shows the dependency of the reflectivity of an uncoated silicon surface on the angle of incidence and the wavelength of the electromagnetic radiation to be reflected, in a three-dimensional representation.

FIG. 3b shows the dependency from FIG. 3a in a two-dimensional representation.

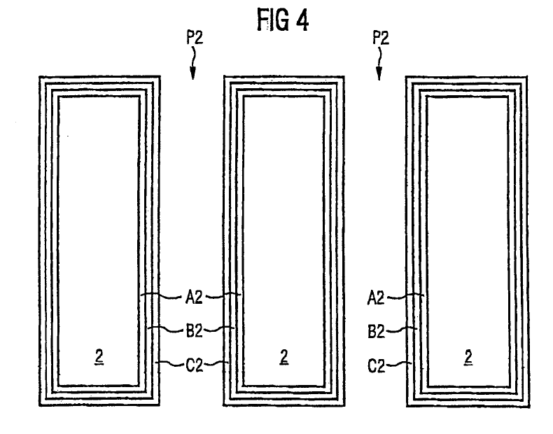

FIG. 4 shows a cross section through a second substrate with a first layer, a second layer and a third layer.

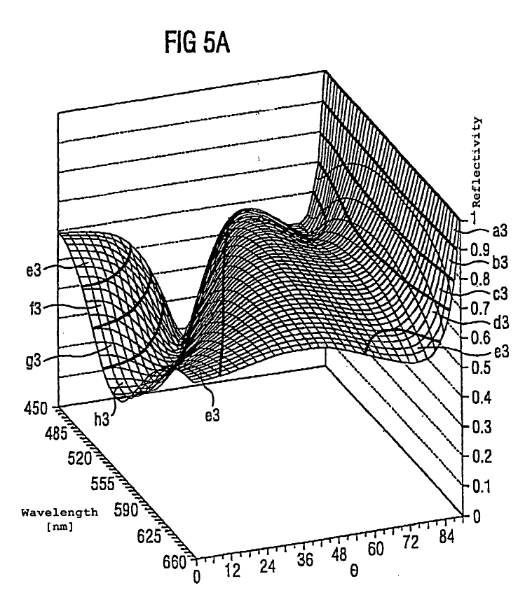

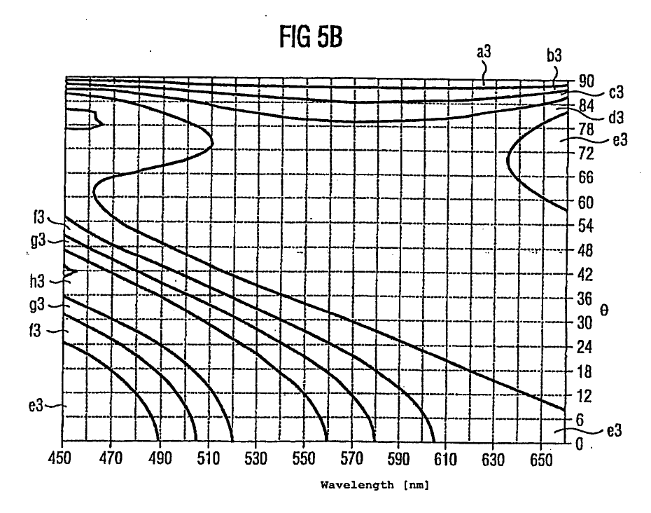

FIG. 5a shows the reflectivity of the coated second substrate as a function of the angle of incidence and the wavelength of the electromagnetic radiation to be reflected, in a three-dimensional representation.

FIG. 5b shows the dependency from FIG. 5a in a two-dimensional representation.

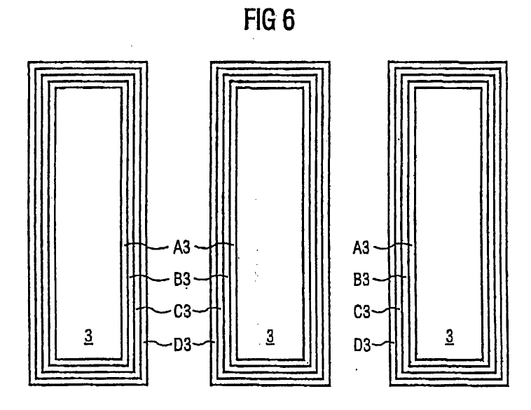

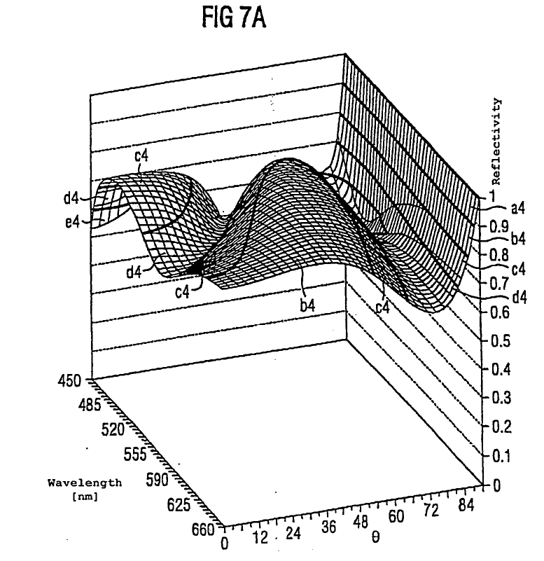

FIG. 6 shows a cross section through a third substrate with a first layer, a second layer, a third layer and a fourth layer.

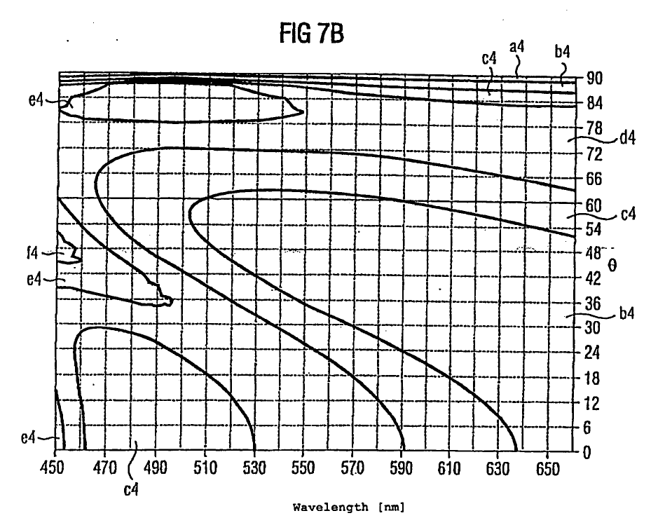

FIG. 7a shows the dependency of the reflectivity of a coated third substrate as a function of the angle of incidence and the wavelength of the radiation to be reflected, in a three-dimensional representation.

FIG. 7b shows the dependency from FIG. 7a in a two-dimensional representation.

FIGS. 1, 4 and 6 are not true to scale.

In a first exemplary embodiment, a first silicon substrate 1, in which pores P that extend from one surface of the first substrate 1 as far as a surface of the first substrate 1 lying opposite the surface are arranged (see FIG. 1), is provided as part of a first biochip. The pores P are approximately 500 μm deep and have a diameter of approximately 10 μm.

Surfaces of the pores and the substrate are provided with a coating for reflecting electromagnetic radiation, which consists of a first layer A1 and a second layer B1 arranged thereon (see FIG. 1). The first layer A1 is approximately 150 nm thick and consists of SiO₂. The first layer A1 made of SiO₂ is produced by thermal oxidation. The second layer B1 is approximately 29 nm thick and consists of polysilicon. The second layer B1 is produced by depositing polysilicon in a CVD process.

FIG. 1 shows the way in which fluorescent molecules can be detected by using the biochip. Stimulating light with a wavelength of approximately 400 nm enters one of the pores P, which has a fluorescent molecule M arranged on its surface provided with the coating. The fluorescent molecule M is stimulated by the stimulating light and emits light with a wavelength of approximately 600 nm with a particular probability in a direction such that the emitted light strikes the surface of the pore P at an angle of incidence θ=70°, and is repeatedly reflected until the emitted light leaves the pore P and can be detected. The thicknesses of the layers A1, B1 are selected in such a way that an optimum reflectivity is achieved for electromagnetic radiation that impinges at an angle of approximately 70° and has a wavelength between approximately 450 nm and 660 nm.

FIGS. 2a and 2b show the reflectivity of the coated first substrate 1 as a function of the angle of incidence and the wavelength of the electromagnetic radiation to be reflected. The reflectivity is the ratio of the intensity of the electromagnetic radiation after reflection to the intensity of the electromagnetic radiation before reflection. Areas that are denoted by a2 exhibit reflectivities between 0.9 and 1 (a2 0.9 to 1). The following likewise apply:

b2=0.8 to 0.9

c2=0.7 to 0.8

d2=0.6 to 0.7

e2=0.5 to 0.6

f2=0.4 to 0.5

g2=0.3 to 0.4

h2=0.2 to 0.3

FIGS. 3a and 3b show the dependency of the reflectivity of a silicon surface without a coating as a function of the angle of incidence and the wavelength of the electromagnetic radiation to be reflected. The following applies for the designation of the areas:

a1=0.9 to 1

b1=0.8 to 0.9

c1=0.7 to 0.8

d1=0.6 to 0.7

e1=0.5 to 0.6

f1=0.4 to 0.5

g1=0.3 to 0.4

h1=0.2 to 0.3

A comparison of FIGS. 2a and 2b with FIGS. 3a and 3b shows that, compared with the uncoated silicon surface, the reflectivity of the first substrate 1 is increased greatly for almost all angles, i.e. for angles of less than approximately 85°, and for wavelengths between 450 nm and 660 nm.

In a second exemplary embodiment, a second biochip with a second substrate 2 is provided, which is configured like the first biochip with the exception that the coating consists of a first layer A2, a second layer B2 arranged thereon, and a third layer C2 arranged thereon (see FIG. 4). The first layer A2 is approximately 185 nm thick and consists of SiO₂. The second layer B2 is approximately 33 nm thick and consists of polysilicon. The third layer C2 is approximately 134 nm thick and consists of silicon nitride. The thicknesses of the layers A2, B2, C2 are selected in such a way that an optimum reflectivity is achieved for electromagnetic radiation that impinges at an angle of approximately 70° and has a wavelength between approximately 450 nm and 660 nm.

Since the wavelengths refer to air, somewhat modified values are obtained during the optimization of the thicknesses of the layers A2, B2, C2 when a medium other than air, for example an aqueous solution, is employed in the pores P2. Similar considerations also apply to the other exemplary embodiments.

A comparison of FIGS. 2a and 2b with FIGS. 5a and 5b shows that the reflectivity of the second substrate 2 is improved, compared with the reflectivity of the first substrate 1, for some wavelengths and angles of incidence. The following applies for the designation of the areas in FIGS. 5a and 5b:

a3=0.9 to 1

b3=0.8 to 0.9

c3=0.7 to 0.8

d3=0.6 to 0.7

e3=0.5 to 0.6

f3=0.4 to 0.5

g3=0.3 to 0.4

h3=0.2 to 0.3

In a third exemplary embodiment, a third biochip with a third substrate 3 is provided, which is configured similarly to the first biochip with the exception that the coating consists of a first layer A3, a second layer B3 arranged thereon, a third layer C3 arranged thereon and a fourth layer D3 arranged thereon (see FIG. 6). The first layer A3 is approximately 191 nm thick and consists of SiO₂. The second layer B3 is approximately 33 nm thick and consists of polysilicon. The third layer C3 is approximately 93 nm thick and consists of silicon nitride. The fourth layer D3 is approximately 27 nm thick and consists of polysilicon. The thicknesses of the layers A3, B3, C3, D3 are selected in such a way that electromagnetic radiation is optimally reflected if it impinges at an angle of incidence of 70° and has a wavelength between 450 nm and 660 nm.

A comparison of FIGS. 5a and 5b with FIGS. 7a and 7b shows that the reflectivity of the third substrate 3 is increased compared with the reflectivity of the second substrate 2. The following applies for the designation of the areas in FIGS. 7a and 7b:

a4=0.9 to 1

b4=0.8 to 0.9

c4=0.7 to 0.8

d4=0.6 to 0.7

e4=0.5 to 0.6

f4=0.4 to 0.5

Many variations of the exemplary embodiments, which likewise lie within the scope of the invention, may be envisaged. For instance, the coating may consist of more than four layers. Other materials may be selected for the layers of the three exemplary embodiments. The thicknesses of the layers of the three exemplary embodiments may be optimized for electromagnetic radiation with other angles of incidence and other wavelengths. The pores of the three exemplary embodiments are also suitable as optical waveguides. In this case, the pores may be curved.

1-14. canceled.

15. A biochip with at least one pore for detecting fluorescent molecules in which the entire side wall of the pores are provided with a coating for reflecting electromagnetic radiation; in which the pores extend from one surface of the substrate as far as a surface of the substrate lying opposite the surface, so that a solution can be pumped through the pores in which the fluorescent molecules are applied to the surface of the pores provided with the coating.

16. The biochip as claimed in claim 15, in which the coating consists of at least two layers arranged above one another.

17. The biochip as claimed in claim 16, in which at least one of the layers consists of SiO₂ polysilicon or silicon nitride.

18. The biochip as claimed in claim 17, in which the coating consists of a first layer which is arranged on the substrate, and a second layer arranged on the first layer.

19. The biochip as claimed in claim 18, in which the first layer consists of SiO₂ or silicon nitride, in which the second layer consists of polysilicon.

20. The biochip as claimed in claim 17, in which the coating consists of a first layer, which is arranged on the substrate, a second layer arranged on the first layer, and a third layer arranged on the second layer.

21. The biochip as claimed in claim 20, in which the first layer and the third layer consists of silicon nitride or SiO₂, in which the second layer consists of polysilicon.

22. The biochip as claimed in claim 17, in which the coating consists of a first layer, which is arranged on the substrate, a second layer arranged on the first layer, a third layer arranged on the second layer, and a fourth layer arranged on the third layer.

23. The biochip as claimed in claim 22, in which the second layer and the fourth layer consist of polysilicon, in which the first layer and the third layer consist of silicon nitride or SiO₂.

24. The biochip as claimed in claim 15, which is at least part of an optical waveguide.

25. The biochip as claimed in claim 15, in which the coating is configured in such a way that it optimally reflects electromagnetic radiation with wavelengths between 400 nm and 700 nm.

26. The biochip as claimed in claim 15, in which the substrate essentially consists of silicon.

27. The biochip as claimed in claim 1, in which the coating contains tungsten.

28. The substrate as claimed in claim 1, with further pores which are configured like the pore which is part of a biochip for detecting fluorescent molecules applied to the surface of the pores provided with the coating.
